(19) United States
(12) Patent Application Publication
(10) Pub. No.: US 2024/0066044 A1
(43) Pub. Date: Feb. 29, 2024

(54) METHODS AND COMPOSITIONS FOR INCREASING NAD+ METABOLOME IN HEALTHY MIDDLE-AGED POPULATION

(71) Applicant: Bioenergy Life Science, Inc., Ham Lake, MN (US)

(72) Inventor: Yongquan Xue, Ham Lake, MN (US)

(21) Appl. No.: 18/261,576

(22) PCT Filed: Jan. 14, 2022
(86) PCT No.: PCT/US22/70209

§ 371 Date: Jul. 14, 2023

Related U.S. Application Data
(60) Provisional application No. 63/137,720, filed on Jan. 14, 2021.

(51) Int. Cl. A61K 31/7004; A61K 31/455

(52) U.S. Cl. CPC A61K 31/7004; A61K 31/455

(57) ABSTRACT

Methods and compositions for increasing NAD levels in human subjects utilizing an effective amount of D-ribose and nicotinamide. The method and composition may also increase glutathione levels in the human subjects. The method and composition may also increase NAD levels and glutathione levels in the human subjects without causing redox unbalance in the subjects.

This application claims the benefit of U.S. Provisional Patent Application No. 63/137,720, filed 14 Jan. 2021, which is incorporated herein in its entirety.

### BACKGROUND

Nicotinamide adenine dinucleotide (NAD+) metabolome, including NAD+, NADH, NADP+, and NADPH, is pivotable for human health, and its decline is correlated with aging and disease as NAD+ is associated with energy production and oxidative stress. NAD+, NADH, NADP+, and NADPH are reusable coenzymes for diverse oxidation-reduction (redox) reactions and energy homeostasis by breaking down and converting nutrients into energy in the form of adenosine triphosphate (ATP). NAD+ and NADP+ are consumable substrates in enzymatic reactions regulating crucial biological processes, including gene expression, energy homeostasis, DNA repair, apoptotic cell death and lifespan, calcium signaling, glucose homeostasis, and circadian rhythms. [1-5] As coenzymes, the NAD+ metabolome participates in over 60% of reactions in cellular metabolism and their homeostasis is the determinant for maintaining redox balance and metabolism. [1,2] As a consumable substrate, NAD+ concentration is directly linked with advancing aging, premature aging [6] and fat composition [7]. NAD-consuming enzymes, including poly (ADP-ribose) polymerase (PARPs), Sirtuins (SIRT1-7), and cADP-ribose synthase (CD38), have far reaching implications for health and disease [8], especially for aging and age-related chronic degenerative diseases.

As represented below, there are four NAD+ biosynthetic pathways operating in mammals [9], including a de novo pathway starting from amino acid tryptophan, and the following three alternative pathways of pyridine salvage:
Pathway #1: de novo biosynthetic pathway from amino acid tryptophan, represented as:

Trp→NAD
Pathway #2: salvage pathway of nicotinamide (Nam), represented as:

NAM+PRPP→NMN+ATP→NAD
Pathway #3: salvage pathway for nicotinic acid (Na), represented as:

NA+PRPP→NAMN+ATP→NAAD→NAD
Pathway #4: salvage pathway for nicotinamide riboside (NR), represented as:

NR+ATP→NAD
Where:
   ATP=adenosine triphosphate
   NA=nicotinic acid
   NAAd=nicotinic acid adenine dinucleotide
   NAD=nicotinamide adenine dinucleotide
   NAM=nicotinamide
   NAMN=nicotinic acid mononucleotide
   NMN=nicotinamide mononucleotide;
   NR=nicotinamide riboside
   PRPP=phosphor-ribose-pyrophosphate
   Trp=tryptophan

In each of the three salvage pathways (Pathways #2-#4) PRPP and/or ATP are needed. It is known that both PRPP and ATP are extension products of D-ribose (i.e., D-ribose+ATP 4 PRPP). The pyridines, NA, NAM and NR are collectively referred to as niacin or vitamin B3 [10] which may arise from dietary intake and/or intracellular NAD+ catabolism. The starting material for the de novo pathway (Pathway #1), tryptophan, is from dietary protein sources such as egg, meat, and cheese. The de novo NAD synthesis is generally considered insufficient to sustain normal NAD homeostasis [11]. The vitamin B3 commonly in enriched food and beverages are also limited in amount because nicotinic acid (NA) causes flushing when its dose is high enough. [12] Most NAD+ in mammals is synthesized from nicotinamide (NAM) via the amidated salvage route. NAM salvage is catalyzed by nicotinamide phosphoribosyl-transferase (NAMPT) [13], which is under regulation of circadian rhythm [4]. Some researchers believe that age-related NAD+ decline is due to NAMPT decline with advancing age. [14] However, additional evidence to enhanced consumption of NAD+ by NAD+ consuming enzymes, such as PARPs, Sirtuins, and CD38 has been reported [15]. More importantly, age-related NAM salvaging capacity in human skeletal muscles can be reserved by exercise. [16] Therefore, it is possible to increase NAD+ by supplementing NAM on a regular basis.

The last decade has witnessed considerable research on enhancing NAD+ by supplementing with NR. [17] NR is a more advanced precursors in the NAD+ biosynthetic salvage pathway. It is believed that NR is converted into NMN by NR kinase (NRK1/2) using ATP as a co-substrate [18]. It has been demonstrated that supplementation with NR increases NAD levels, enhances oxidative metabolism, and reduces fat and hepatic steatosis. [17, 18] There are, however, controversies over its benefits. Firstly, multiple experiments demonstrate that NR degraded very quickly into nicotinamide (NAM) and ribose, especially when ingesting orally. [19, 20] Therefore, the reported benefits of NR are likely attributable to circulating nicotinamide or nicotinamide and ribose. Secondly, supplementing NR to healthy subjects reduces their exercise performance [21, 22], which may limit its use in a healthy active population. To address these issues and to overcome these shortcomings of NR, Applicant has initiated research investigation using different combinations of nicotinamide and D-ribose.

Nicotinamide (NAM) is the preferred treatment for pellagra. [23] NAM is also used to for acne and non-melanoma skin cancer. [24] More recently, NAM is considered as a potential candidate to increase the NAD+ metabolome for anti-aging applications. [25] High-dose NAM indeed enhanced NAD+ levels and ameliorated disease in a rat model of obesity [26]. When considering its long-term application, Applicant identified several limiting factors. The daily recommended dietary intake to prevent vitamin B3 deficiency is only approximately 15 mg in adults. [27] Doses in excess of 3 g per day can cause side effects including hepatotoxicity. [28] The daily tolerable upper dietary intake of NAM is specified accordingly at 900 mg in the EU (Opinion of the Scientific Committee on Food on the Tolerable Upper Intake Levels of Nicotinic Acid and Nicotinamide, issued by Scientific Committee on Food), 500 mg in Canada, and 5 mg/kg in Japan (Overview of Dietary Reference Intakes for Japanese, issued by Ministry of Health, Labor and Welfare). Therefore, it is only practical to use NAM at a relatively low dose range, especially 100-500 mg per day. It is also important to note that NAM is ineffective for boosting NAD+ at doses below 90 mg per day. Beyond 900 mg, it poses a regulatory challenge. Therefore, maximizing its NAD+ boosting capability in a low dose range is preferable.

Building on data of Applicant's prior preclinical animal studies to determine the pharmacodynamics and tissue distribution of NAD+ metabolites as disclosed in International Patent Application No. PCT/US2019/031889 (Publication No. WO2019/217935), which is incorporated herein in its entirety by reference, Applicant has developed a novel combination of NAM and D-ribose that amplifies the NAD-boosting capability of NAM and reduces its potential side. This novel combination of NAM and D-ribose is distributed under the trademark RiaGev® available from Bioenergy Life Science, Inc., 13840 Johnson Street NE, Ham Lake, MN USA 55304.

As disclosed herein, Applicant conducted clinical trials with the RiaGev product under a randomized, triple-blind, comparator-controlled, cross over pilot study that investigated the efficacy and safety of RiaGev via evaluation of the NAD+ metabolome and diverse health related parameters in healthy adults age between the age of 35 to 65. The clinical trial confirmed that the supplementation with the RiaGev product increased the NAD+ metabolome of the subjects and that it was safe and effective in preventing redox unbalance caused by exhaustive aerobic exercise of healthy, active, midlife stage human subjects.

### BRIEF DESCRIPTION OF THE DRAWINGS

FIG. 1 is a diagram of the clinical trial.

FIG. 2 is a chart summarizing the number of participants screened, the number of screen failures and reasons, the number of randomized participants, and the grouping of the participants.

FIG. 3 is a table of the mean and median data of the clinical assessments of the participants in the Comparator-IP Group and in the IP-Comparator group.

FIG. 4A is a graph of the NAD+ primary outcome of the Comparator Group and RiaGev Group.

FIG. 4B is a graph of the NADP+ primary outcome of the Comparator Group and RiaGev Group.

FIG. 4C is a graph of the NAD+ and NADP+ level change over time of the Comparator Group and RiaGev Group.

FIG. 5 is a graph of the total serum ATP and ADP of the Comparator Group and RiaGev Group.

FIG. 6 is a graph of the total serum Glutathiones of the Comparator Group and RiaGev Group.

FIG. 7 is a graph of the salivary cortisol of the Comparator Group and RiaGev Group.

FIG. 8A is a graph of the OGTT blood glucose on day 8 versus day 1 of the RiaGev Group.

FIG. 8B is a graph of the OGTT insulin on day 8 versus day 1 of the RiaGev Group.

FIG. 8C is a graph of the OGTT blood glucose on day 8 versus day 1 of the Comparator Group.

FIG. 8D is a graph of the OGTT insulin on day 8 versus day 1 of the Comparator Group.

FIG. 9A is a graph of the NADPH/NADP+ change in the Comparator Group and RiaGev Group before and after exercise at 1 day 1 and day 8.

FIG. 9B is a graph of the GSH/GS SG change in the Comparator Group and RiaGev Group before and after exercise at day 1 and day 8.

FIG. 10A is a graph of the CIS physical fatigue of the Comparator Group and RiaGev Group from day 1 to day 8.

FIG. 10B is a graph of the CIS concentration of the Comparator Group and RiaGev Group from day 1 to day 8.

FIG. 10C is a graph of the CIS motivation of the Comparator Group and RiaGev Group from day 1 to day 8.

FIG. 10D is a graph of the CIS total score of the Comparator Group and RiaGev Group from day 1 to day 8.

### DESCRIPTION

A randomized, triple-blind, comparator-controlled, cross over study investigated the efficacy and safety of a nicotinamide (NAM) and D-ribose composition (sold under the trademark RiaGev®), via evaluation of NAD+ metabolome and health related parameters in healthy adults of ages 35-65. The trial was approved by IntegReview, Internal Review Board (protocol code 19RNHB(1918)) and the study is registered on ClinicalTrials.gov under the identifier NCT04483011. [33]

The midlife stage for the test subjects was selected because many health problems that directly affect healthy aging occur during this period. Also, this is the period in life that bears the heaviest burden of stress. Oxidative stress is a known factor for many chronic diseases and is detrimental to healthy aging. [29, 30] Two of the most common chronic diseases that are accompanied by aging are obesity and diabetes. In 2016 the World Health Organization (WHO) reported that approximately 1.6 million deaths were attributed to diabetes. Half of these individuals had high blood glucose before the age of 70 (3). Hence it is crucial to actively control blood glucose and oxidative stress during one's midlife stage. Therefore, stress parameters and blood glucose are secondary outcomes following the primary outcome of NAD+ metabolome of the clinical trial.

### Subject Population

Subjects for the study were healthy, active, male and females between the ages of 35 and 65 years of age. The major inclusion criteria for the subjects included the following: Subjects having a body mass index (BMI) between 18.5 to 29.9 kg/m2; female subjects were not child-bearing; subjects were healthy as determined by laboratory results, medical history, physical exam and EKG; the subjects agreed to avoid supplementation with tryptophan and vitamin B3 or its derivatives (niacin, nicotinic acid, niacinamide) one week prior to randomization and during the study; the subjects had the ability to complete maximal and submaximal exercise tests; the subjects agreed to maintain current diet, activity level, and sleeping cycle throughout the study; the subjects agreed to comply to all study procedures with voluntary, written, informed consent to participate in the study. The study excluded subjects with any diseases or inflammatory conditions. The detailed inclusion and exclusion criteria are listed in ClinicalTrials.gov under the identifier NCT04483011. [33]

### Investigational Product and Comparator

The investigational product (IP), RiaGev®, contained 1280 mg of D-ribose, 240 mg of nicotinamide, and 480 mg of palm oil, packed into three capsules. D-ribose and nicotinamide are active ingredients and palm oil is an excipient. The batch number for the RiaGev was S0776313. The Comparator, contained 1280 mg dextrose and 480 mg palm oil, also packed in three capsules of same size and color as the IP. Dextrose is used to match the calories of the D-ribose in the IP. The batch number for the Comparator was S1126314. Both the IP and the Comparator were provided by Bioenergy Life Science, Inc., 13840 Johnson Street NE, Ham Lake, MN USA 55304.

### Screening and Assignment of Participants to Groups

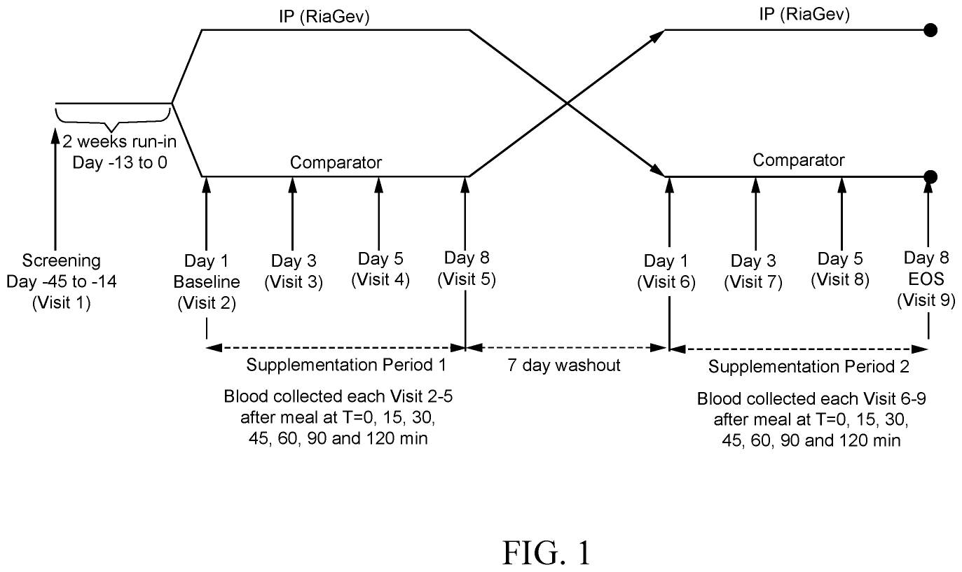

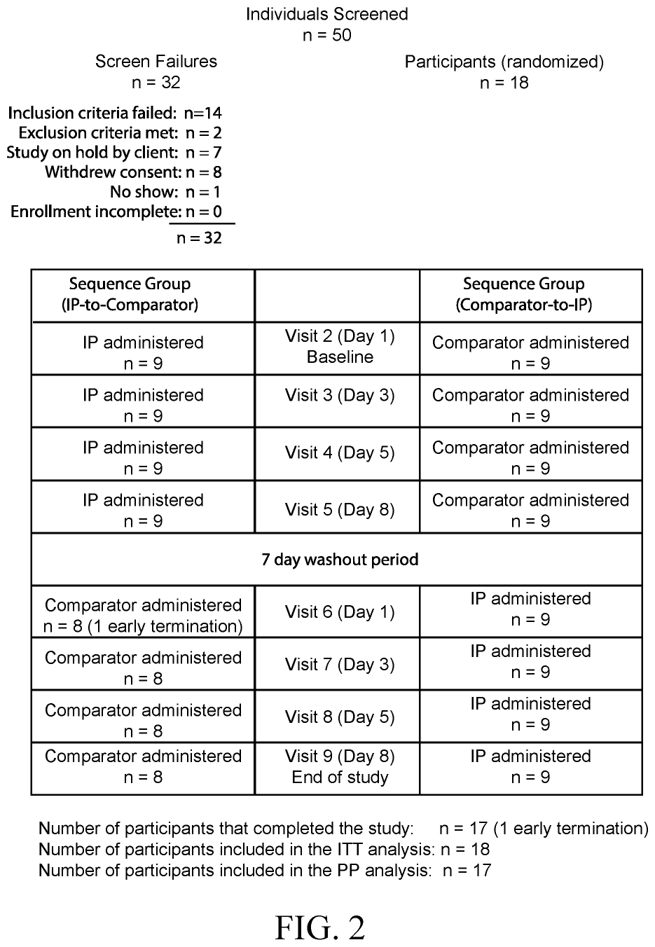

Referring to FIGS. 1 and 2, a total of 50 healthy men and women between the ages of 35 and 65 years were screened as potential participants for the clinical trial. All potential participants were identified by their initials and their date of birth and each was assigned a participant number at the initial screening visit (Visit 1). Out of the 50 individuals screened, 18 individuals qualified as meeting all the inclusion criteria and not meeting any of the exclusion criteria. The 18 qualifying individuals were recruited for the study. At Visit 2 (baseline), each of the 18 individuals (hereinafter referred to as the participants) was assigned a randomized number by a blinded investigator. The randomized participant number was generated via a randomized list generator.

The 18 participants were then randomized into two matching groups of 9 participants each, respectively referred to as the “IP-to-Comparator Group” and the “Comparator-to-IP Group”, based on their demographic and physical information, such as age, sex, weight and height. The BMI, heart rate, and hemoglobin A1c of each group were also not statistically different.

### Administration

All participants were instructed to take two doses of the capsules daily, once in the morning and once in the evening. Each dose was 3 capsules, one dose was administered immediately before breakfast and the other immediately before dinner. In both supplementation periods, on Day 1 only the evening dose was administered and on Day 8 only the morning dose was administered. The participants were instructed to save all unused and open packages of the capsules and to return them for a determination of compliance. If a dose was missed, participants were instructed to consume the missed dose anytime on the same day, except at bedtime. Participants were advised not to exceed two doses daily.

During each supplementation period, one group received two daily doses of the IP capsules while the other group received two daily doses of the Comparator capsules. After the first supplementation period, all participants washed out for 7 days, then crossed over to take the capsules of the other product. For clarity, the participants in the IP-to-Comparator Group would first be administered the IP capsules during Supplementation Period 1 of the study (i.e., first set of Days 1-8). After the 7 day washout period the IP-to-Comparator Group would then be administered the Comparator capsules during Supplementation Period 2 of the study (i.e., second set of Days 1-8). Conversely, the participants in the Comparator-to-IP Group would first be administered the Comparator capsules during Supplementation Period 1 of the study (i.e., first set of Days 1-8). After the 7 day washout period the Comparator-to-IP Group would be administered the IP capsules during Supplementation Period 2 of the study (i.e., second set of Days 1-8).

### Blinding

The clinical trial was a triple-blind study conducted by the Prism Clinical Research, Minneapolis, MN. The IP and the Comparator were sealed in identical packages, each labelled per the requirements of ICH-GCP guidelines and applicable local regulatory guidelines. Un-blinded personnel at Prism Clinical Research who were not involved in any study assessments labelled the IP and Comparator packages. A randomization schedule was created and provided to the Prism Clinical Research investigators indicating the order of randomization. All Prism Clinical Research investigators, including the principal investigator and other on-site personnel, as well as the participants, were blinded with respect to the IP and the Comparator.

### Clinical Assessments, Blood Collection and Analysis

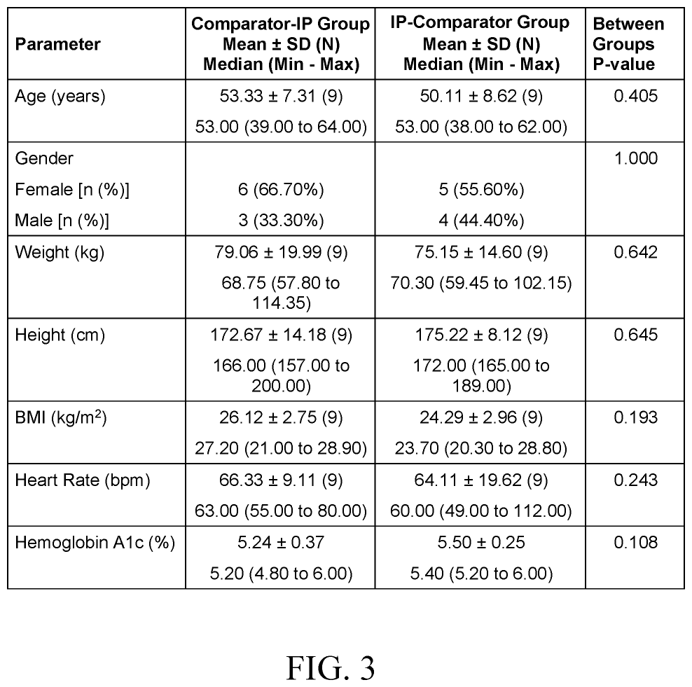

Prism Clinical Research measured the height, weight, blood pressures, and heart rates of the participants using standard procedures. For participants of childbearing capacity, Prism Clinical Research conducted urine pregnancy tests (Henry Schein One Step+) at Visits 1 and 2. A table of the participants' mean and median measured parameters and clinical assessments are shown in FIG. 3 for each of the IP-Comparator Group and Comparator-IP Group.

Prism Clinical Research collected blood samples of each participant at Visits 1-9 for analysis using the following procedures: 1) Heparin plasma tubes were used for whole blood collection (BD vacutainer, sodium heparin 95 USP Units, REF 367878); 2) Blood was drawn, gently mixed by inverting the tube 5 to 6 times, and quickly make 400 uL (accurately measured) aliquots of blood using 2.0 mL pre-cooled Eppendorf tubes (see the catalogue number below) at 4° C. on ice; 3) Immediately, the aliquots were frozen using a dry ice bucket and then transferred to −80° C. freezer; and 4) the frozen aliquots were shipped over dry ice to Northwest Metabolomics Research Center, University of Washington, Seattle, Washington, USA, for analysis of the coenzymes. Enough dry ice was packed in the shipping box to ensure samples remained frozen until received.

The safety endpoints of complete blood count (WBC count with differential, RBC count, hemoglobin, hematocrit, platelet count, RBC indices (MCV, MCH, MCHC, RDW)), liver function (AST, ALT, bilirubin), and kidney function laboratory blood tests (creatinine, eGFR, electrolytes) were analyzed from the blood drawn at Visits 1 (screening), 5, 6, and 9 by HCMC Pathology Lab, Minnesota, USA, using standardized procedures. At visits 2, 5, 6 and 9, glucose and insulin were analyzed also by HCMC Pathology Lab using standardized procedures.

Northwest Metabolomics Research Center, analyzed Glutathione (GSH), Glutathione disulfide (GSSG), adenosine triphosphate (ATP), adenosine diphosphate (ADP), and adenosine monophosphate (AMP) using established NMR methodologies at visits 2, 5, 6 and 9. At visits 2-9, NAD+, NADP+, and NADPH, were analyzed using established NMR methodologies [31] also by Northwest Metabolomics Research Center.

### CIS Questionnaire

The standard Checklist Individual Strength (CIS) Questionnaire was used in the study. The CIS Questionnaire contains 20 questions scored on a 7-point scale measuring subjective fatigue experience, reduced concentration, reduced motivation and reduced physical activity level. [32] The questions and scoring method follow the reference. The CIS Questionnaire was administered to participants by Prism Clinical Research on Visits 2 to 8.

### Saliva Collection

Participants collected salivary cortisol samples using the Salivette collection device. Saliva samples were collected within 15 minutes of waking and prior to eating on the mornings of all visits, except screening (Visit 1). To ensure proper collection, participants were provided instruction by Prism Clinical Research.

### Treadmill Exercise Data Collection

To determine the participant's maximum heart rate, at each participant's initial screening (Visit 1), Prism Clinical Research conducted a graded treadmill test following a ramped Bruce protocol. Participants continued to advance stages until volitional exhaustion by the participant. Throughout the test, the participant's heart rate was monitored and recorded with a chest strap heart rate monitor. Successful competition of the test was achieving 85% or greater of age predicted maximum heart rate (220-age).

Each participant performed additional treadmill exercises on Visit 2 (Period 1, Day 1), Visit 5 (Period 1, Day 8), Visit 6 (Period 2, Day 1), and Visit 9 (Period 2, Day 8). On the day of the treadmill exercise, participants were instructed to warm up on the treadmill first for five minutes at a leisurely walking pace. When the participants were ready, speed was increased to 60% of participant's max HR with 5% grade incline and they were instructed to walk on the treadmill for 30 minutes or until exhaustion.

### Food Records

Participants were asked to record their food consumption during the study. The participant's food records were used to calculate and analyze their daily calories, macronutrient and micronutrient intake throughout the study using Nutritics software (Nutritics, 2019). The food records were reviewed by trained staff at Prism Clinical Research at each of the participant's visits. The participants were counseled by the Prism Clinical Research staff with dietary suggestions as required. All participants were provided with instructions on how to complete their food records.

### Compliance

Each participant's compliance to the study procedures was recorded by Prism Clinical Research staff in the relevant section of the compliance report from at each visit. Each participant's compliance with the administration of the IP and Comparator was assessed by counting the returned, unused IP and Comparator capsules at each visit. Compliance was calculated by determining the number of dosage units taken divided by the number of dosage units expected to have been taken multiplied by 100.

### Recordation of Adverse Events

During the study, each participant recorded any adverse events (AEs) in a diary. At each visit, the participant was asked: “Have you experienced any difficulties or problems since I last saw you?” Any AE noted by the participant was documented in the study record and was classified as per the description, duration, intensity, frequency, and outcome. The principal investigator at Prism Clinical Research assessed the AEs and decided causality.

### Statistical Analyses

The following analytical populations were defined for this study: The Intent-To-Treat (ITT) Population and the Per Protocol (PP) Population. The ITT population consisted of all participants who received either product, and on whom any post-randomization effectiveness information was available. The ITT population was used to present all the effectiveness information according to the treatment to which subjects were randomized. The PP Population consisted of all participants who consumed at least 80% of the IP and Comparator doses, did not have any major protocol violations and completed all study visits and procedures connected with measurement of the primary variable.

For categorical variables, counts and percentages were presented. The denominator for each percentage was the number of subjects within the study group unless otherwise specified. Possible differences between groups were assessed by using two-tailed Chi-square or Fisher's exact test, as appropriate.

For summaries of interval variables, the arithmetic means, standard deviation, median and minimum-maximum range were presented to two decimal places. These were accompanied by the number of participants included in the analysis for that time point. Possible differences between groups at screening/baseline visits were assessed by ANOVA. For each group, change in each outcome between study time points was assessed using a paired Student's t-test if normally distributed or Wilcoxon Signed-Rank test if otherwise.

Changes in continuous endpoints from screening/baseline were calculated as:

Change to Ti=Value at Ti−Value at Tscreening/baseline

Change in the primary outcome and each secondary outcome was compared between groups using repeated measures mixed model analysis of covariance (ANCOVA) if normally or log-normally distributed. Each model included the study group* time (study visit) as a fixed effects, the baseline value of the dependent variable as a covariate and subject as random effect. Between group P-values were obtained from the final model.

A descriptive analysis was provided for pre-emergent and post-emergent adverse events (AEs) reported in this study. Furthermore, the outcome and relationship to study products was reported each AE that was classified as possibly or probably related to the study products. The number of participants with at least one AE was compared between study arms using the Fisher's Exact test. Vital signs, hematology, and clinical chemistry parameters were summarized as means, standard deviation, median, and minimum-maximum range. Changes from screening/baseline were assessed using the paired t-test.

All hypothesis testing was carried out at the 5% (2-sided) significance level unless otherwise specified. P-values were rounded to three decimal places. P-values less than 0.001 were reported as <0.001 and those less than or equal to 0.05 were considered statistically significant. All analyses were performed using R Statistical Package version 3.6.3 (R Core Team, 2020) for Microsoft Windows.

Results—Primary Outcome-NAD+ metabolome

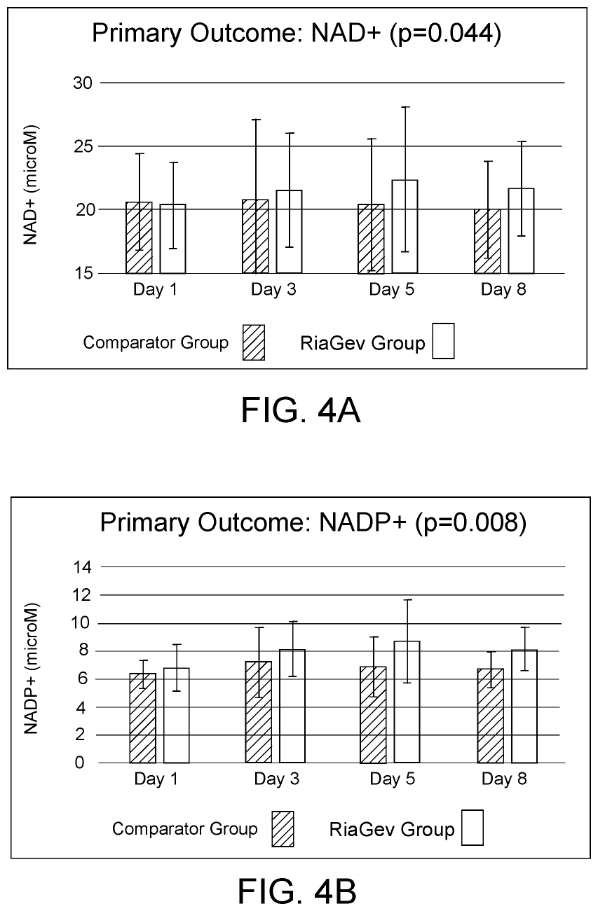

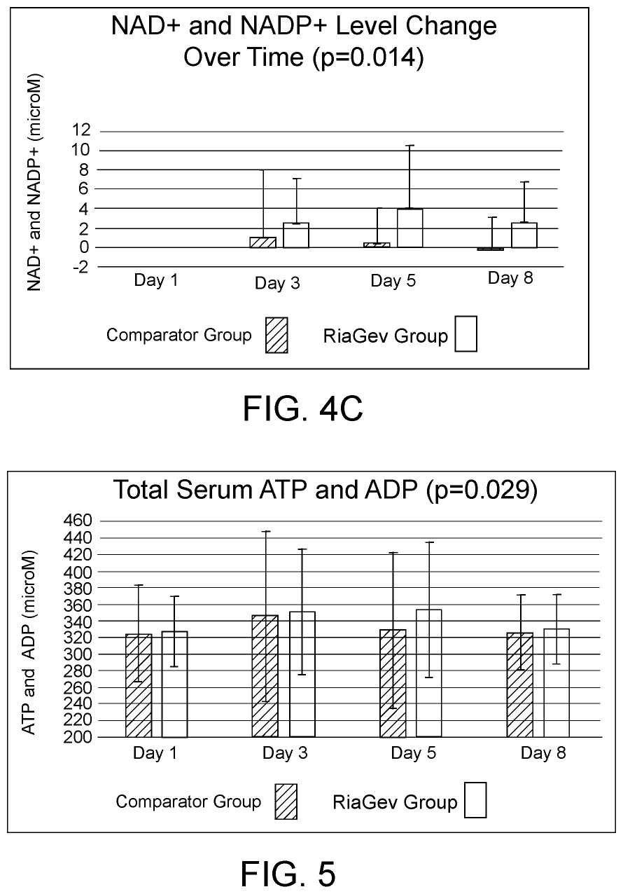

The primary outcome of this study is NAD+ metabolome, especially NAD+ level, after supplementing with the IP (the “RiaGev Group”)) in comparison to supplementing with the Comparator (the “Comparator Group”). As shown in FIG. 4A, NAD+ levels in the RiaGev Group increased steadily over the baseline (Day 1) after supplementation. At day 5, NAD+ concentration in the RiaGev Group is significantly more than the baseline, a 10.4% increase (p=0.034), which is also significantly more than the Comparator Group (p=0.044). At Day 8, there is also a trending significant increase of 6.4% in NAD+ level over baseline (p=0.07). By comparison, the NAD+ level in Comparator Group did not change significantly over the period.

Comparing with the mild increase in NAD+ level, we have noticed an unexpectedly large increase in NADP+ level (FIG. 4B). Significant within-group increases of 19.1%, 27.6% and 19.6% were recorded with RiaGev at Days 3, 5 and 8 over baseline respectively (p≤0.008). The NADP+ concentration for the RiaGev Group is also significantly greater than Comparator Group (p≤0.040).

Referring to FIG. 4C, when combined, NAD+ and NADP+ concentrations observed at Day 3, 5 and 8, were significantly greater in the RiaGev Group than in the Comparator Group at each day (p≤0.029). In conjunction with significant within-group concentration increases of 9.4%, 14.8% and 9.7% reported with the RiaGev Group at Days 3, 5 and 8 respectively (p≤0.032).

In contrast to NAD+ and NADP+, the NADPH level did not change significantly over the study period, except at Day 1, where a significant within-group decrease in NADPH concentration after exercise with the Comparator (p=0.039). The NADH level was not measured in this study because the preservative used during shipment destroys its NMR signal. Whole blood 1-methyl-nicotinamide (MeNAM) and nicotinic acid adenine dinucleotide phosphate (NAAD(P)) were below the detection limit in the blood samples.

In summary, comparing with the baseline (Day 1) and the Comparator Group, the NAD⁺ concentration in the blood of RiaGev Group on Day 5 was 10.4% more over the baseline (p=0.034) and over the Comparator Group (p=0.044). The NADP⁺ concentration of the RiaGev Group on Day 5 was 27.6% more over the baseline (p=0.007) and over the Comparator Group (p=0.033). The combined NAD⁺ and NADP⁺ concentration of the RiaGev Group on Day 5 was 15% over baseline (0.004) and over the Comparator Group (p=0.014).

### Results—Secondary Outcomes

ATP is the universal carrier for energy. FIG. 5 shows the total serum ATP and ADP levels between Day 1 and Day 8 for the Comparator Group and RiaGev Group. The total ATP and ADP level is reported because the sum is more accurately measured than ATP alone in the NMR analysis. The total high energy phosphates (ATP and ADP) is significant more in the RiaGev Group than in the Comparator Group at Day 5 (7.3% higher, p=0.029).

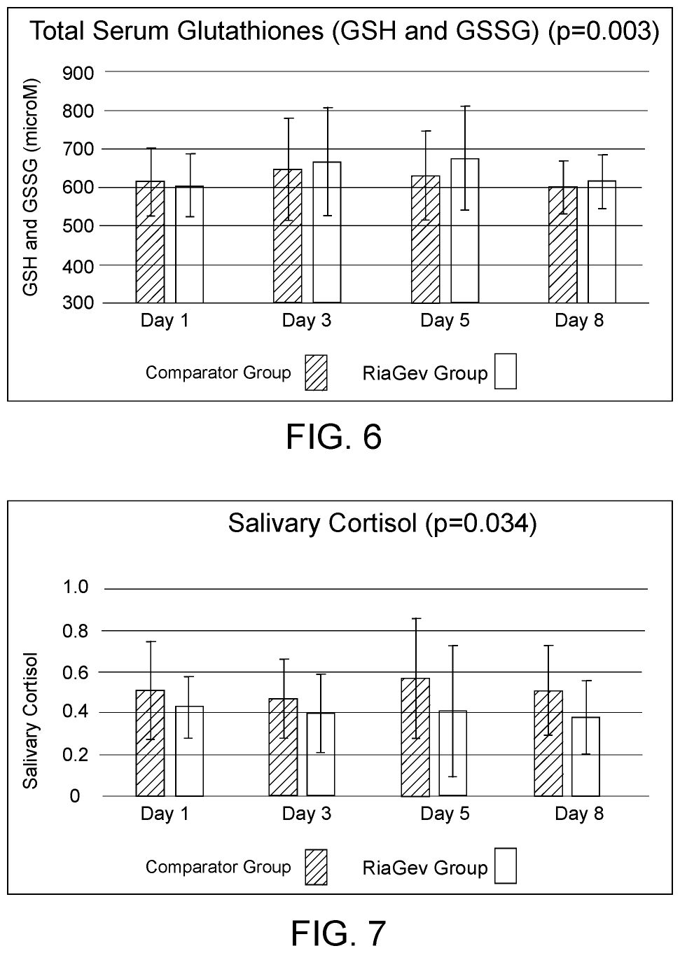

Glutathione is a circulating antioxidant generated in the body. Both reduced (GSH) and oxidized (GSSG) glutathione were measured in the blood. The total serum glutathione (GSH+GSSG) concentration in blood is presented in FIG. 6. There was a significant increase of total glutathione after RiaGev supplementation. At Day 3 and 5 total glutathione concentration in the RiaGev group is 10.2% and 11.6%, higher than at day 1, respectively (p<0.016). The Comparator Group had no significant change in total glutathione during the same period. In summary, total serum glutathione of the RiaGev Group on Day 5 was 11% more than baseline (Day 1) and over the Comparator Group (p=0.003)

The waking salivary cortisol is presented in FIG. 7. In the RiaGev Group, cortisol level steadily declined after Day 1 as supplementation continued, while the cortisol level fluctuated in the Comparator Group throughout the study period. There was a significant between-group difference in waking salivary cortisol at Day 5 and Day 8, where the RiaGev Group had a significantly lower level of cortisol than the Comparator Group (p<0.044).

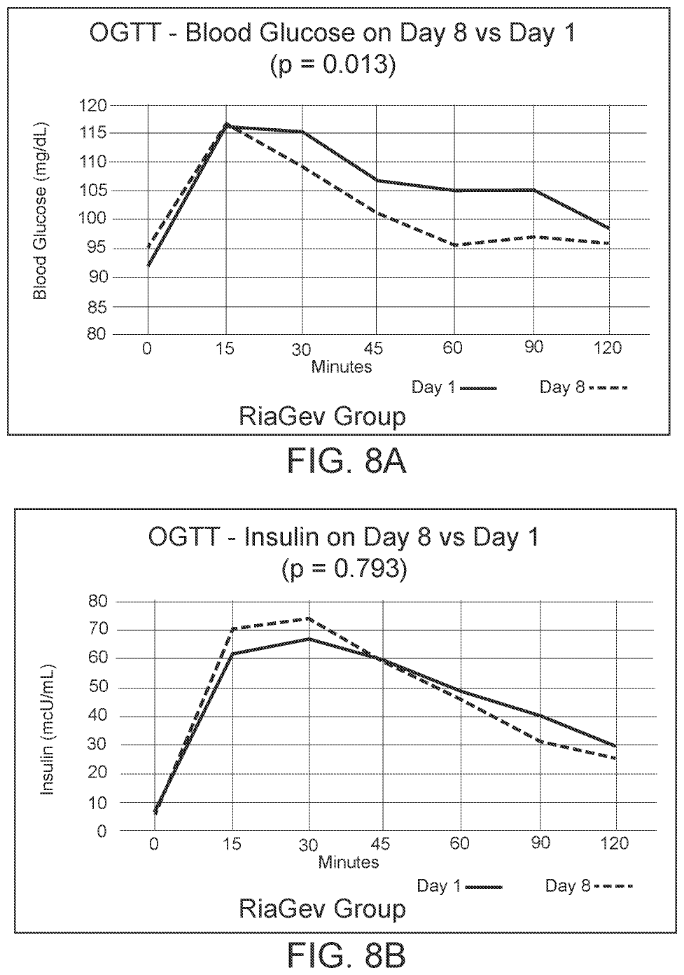

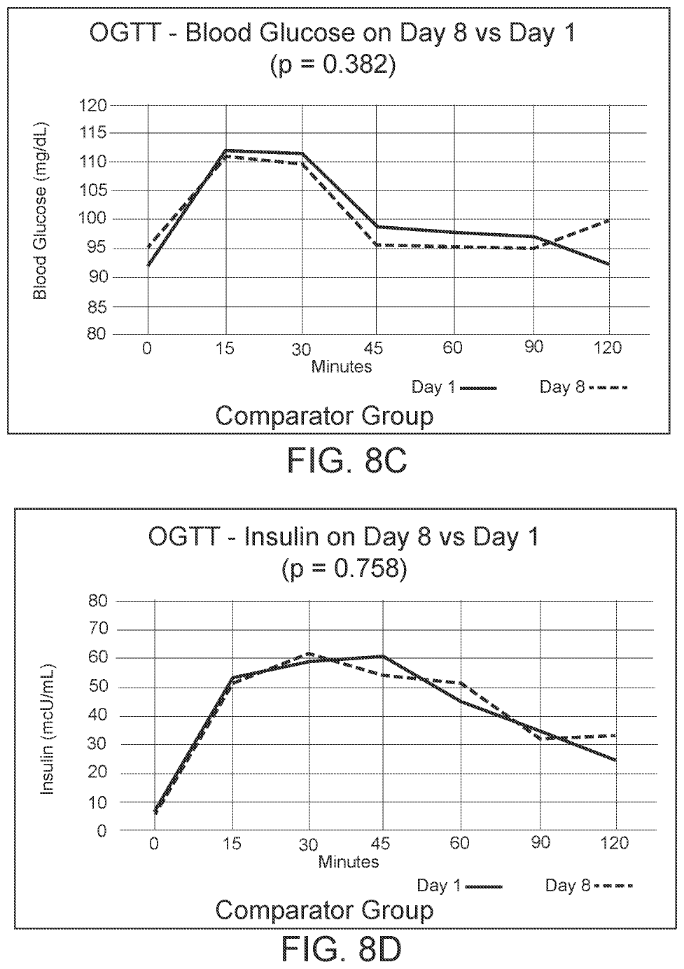

Post-prandial oral glucose tolerance test (OGTT) were performed to determine the blood glucose and insulin response to a standardized meal before and after 7-day supplementation with RiaGev. FIGS. 8A and 8B show blood glucose and insulin levels for the RiaGev Group, respectively, post prandial at Day 1 (before supplementation) and Day 8 (after 7-day supplementation). For the RiaGev Group, the overall blood glucose (incremental Area Under the Curve, iAUC) at Day 8 was significantly reduced over Day 1 (61% reduction, p=0.013). However, the overall insulin (iAUC) in OGTT on Day 8 was not significantly different from at Day 1 (p=0.793). On the other hand, the insulin peak on Day 8 was higher than on Day 1 (74 vs 67 mcU/mL, respectively), but the glucose peak remains the same (116 vs 114 mg/dL at 15 min post prandial at Day 8 vs Day 1, respectively). As a result, the glucose concentration at Day 8 dropped more rapidly than at Day 1. FIGS. 8C and 8D show blood glucose and insulin levels for the Comparator Group, respectively, post prandial at Day 1 (before supplementation) and Day 8 (after 7-day supplementation). The blood glucose and insulin profile for the Comparator group were not much different on Day 8 vs Day 1.

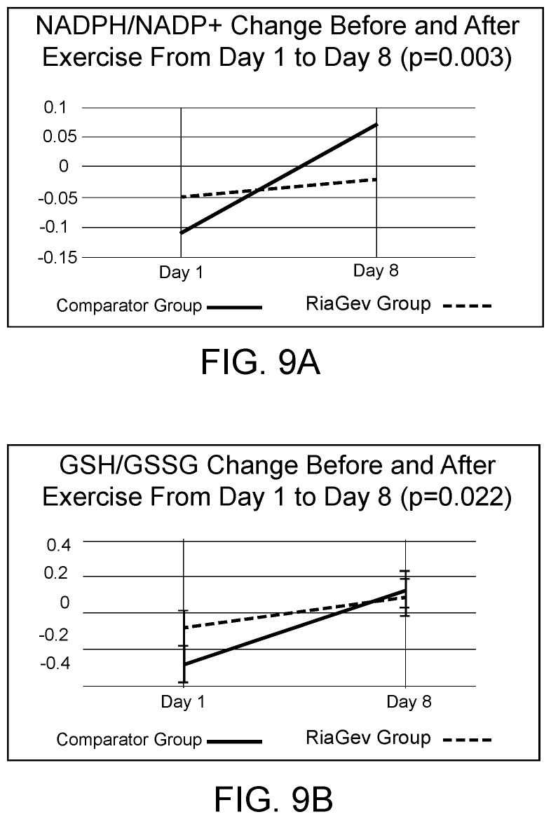

FIGS. 9A and 9B show, respectively the NADPH/NADP+ and GSH/GSSG of the Comparator Group and RiaGev Group before and after exercise from Day 1 to Day 8. At Day 1 (before supplementation) and Day 8 (after supplementation), after the treadmill exercise was conducted with each participant. The inclined and speed of the treadmill is increased stepwise until the subject reaches his or her 60% VO2 max and the subject continues at that pace until exhaustion. Blood samples were collected immediately before and after the exercise to be analyzed for redox balance, including NADPH/NADP+ and GSH/GSSG, and energy charge ATP/AMP. A change is defined as a measurement after the exercise minus before exercise. NADPH/NADP+ and GSH/GSSG ratios decreased significantly at Day 1, especially in the Comparator Group (p=0.003, and p=0.022, respectively), indicating oxidative redox disturbance by the submaximal exercise regimen. This redox perturbation is prevented by supplementation with RiaGev, keeping the redox ratios unchanged during exercise at Day 8. The Comparator caused a none significant redox increase which is expected from functionality of glucose in the Comparator.

As shown in FIG. 9A, the NADPH/NADP+ ratio significantly decreased (p=0.004) after exercise at Day 1 in the Comparator Group, indicating that submaximal exercise significantly disturbs a subject's redox balance. This oxidative disturbance was prevented by the 7-day RiaGev supplementation as shown on Day 8, where NADPH/NADP+ ratio remains relatively unchanged before and after exercise. It is interesting to note that the Comparator supplementation increased the NADPH/NADP+ and GSH/GSSG ratios slightly. This is consistent with functionality of the glucose ingredient (dextrose) in the Comparator.

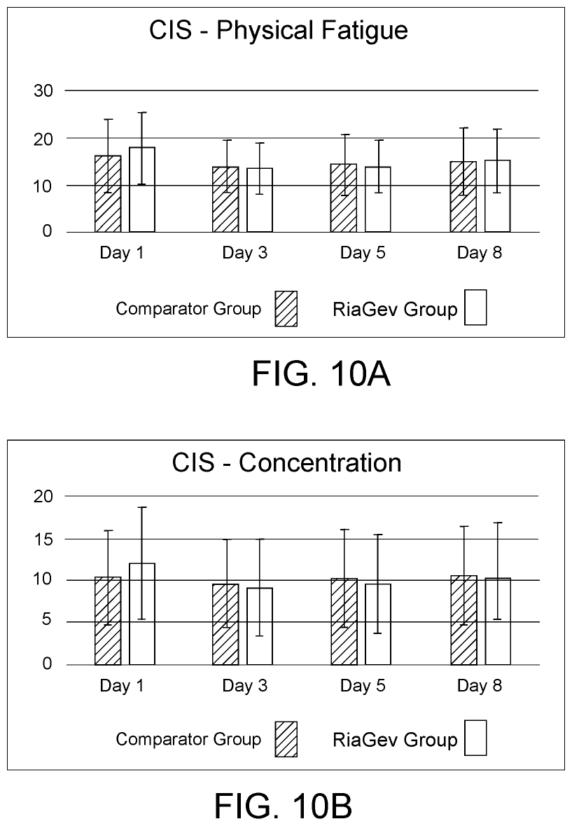

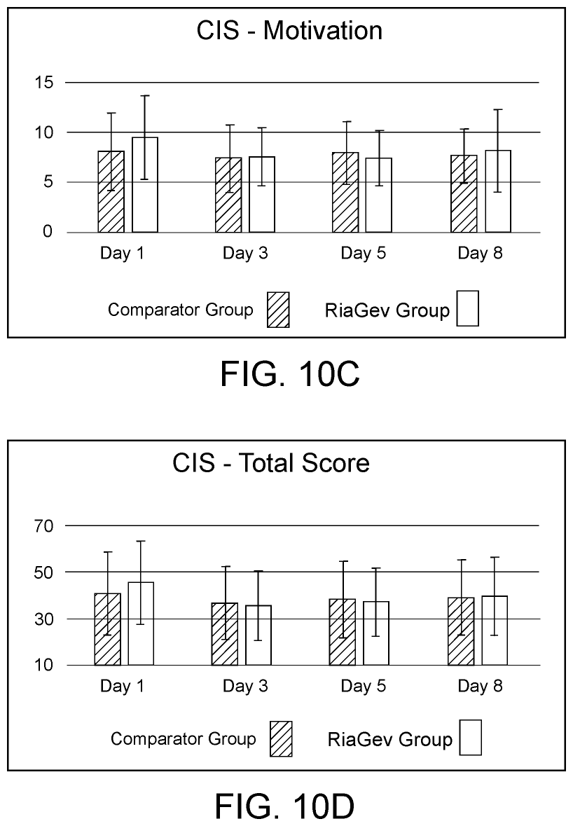

The Checklist Individual Strength (CIS) questionnaire containing a standard set of 20 questions with subscales in physical fatigue, mental concentration, motivation, and physical activities. [31] The CIS total score (FIG. 10D) represents physical and mental tiredness, with four subscales reflecting physical fatigue (FIG. 10A), concentration (FIG. 10B), motivation (FIG. 10C), and physical activity (not shown). Both the RiaGev Group and the Comparator Group showed improved quality of life scores during the study. However, the improvement in the RiaGev Group was consistently greater than the Comparator Group in all sub scales. Specifically, at Day 3, 5, and 8, the total CIS scores were improved by 21.5% (p=0.04) vs 10.4% (p=0.07), 18.3% (p=0.014) vs 6.2% (p=0.049), and 12.7% (p=0.15) vs 4.1% (p=0.361) in the RiaGev Group vs the Comparator Group, respectively. However, the difference between the two groups did not reach significant level (p=0.224).

For the subscales, physical fatigue (FIG. 10A) showed the biggest improvement over baseline and biggest difference between the groups. At Day 3, 5, and 8, the physical fatigue improved 24.3% (p=0.003) vs 13.6% (p=0.041), 21.2% (p=0.009) vs 11.6% (p=0.08), 15.1% (p=0.132) vs 7.4% (p=0.17) in the RiaGev Group vs the Comparator Group, respectively. Referring to FIG. 10B, concentration in the RiaGev Group also improved significantly at 22.9% (p=0.014), 19.8% (p=0.012) and 14.3% (p=0.118) at Day 3, 5, and 8, respectively, whereas, the improvement in Comparator group is less significant at any of those days. The same trend is true for motivation (FIG. 10C), where the RiaGev Group improved by 20.4% (p=0.13), 22.2% (p=0.015), and 14% (p=0.163) at Day 3, 5, and 8, respectively, whereas in the Comparator Group, statistically significant improvement. Among the subscales tested, the physical activity scale (not shown) did not improve in either RiaGev Group or Comparator Group during the trial periods. This is expected because a 7-day supplement period is not long enough to see behavioral change.

No clinically relevant changes in physical measurements, vital signs, hematology, kidney markers or electrolytes were observed from pre- to post-supplementation of the participants in this study. All participants were deemed healthy by the principal investigator after both treatment periods.

A total of 12 post-emergent adverse events (AEs) were reported by 10 participants in this study. Of these, nine were reported by seven participants while taking RiaGev and 3 by 2 participants while taking the Comparator. None of the post-emergent AEs were categorized as ‘most probable’ in terms of relation to the product. Two AEs, weakness and loss of appetite, were categorized as ‘possible’ with RiaGev and one of joint pain with the Comparator. All AEs were resolved by the end of the study. The principal investigator assessed all subject as healthy before and after the trial.

### Observations

It was discovered that RiaGev® primarily enhances NADP+ instead of NAD+(27% vs 11% increase in the study) is unprecedented in NAD+ boosting precursors. NADP+ is a more advanced product than NAD+, which requires an additional high energy phosphate for its generation. Therefore, the higher NADP+ yield means that the body is at higher energy status. This is consistent with an increase in high energy phosphates (ATP and ADP) and glutathione in the circulating blood. It is also consistent with less fatigue, improved concentration and motivation as reported by the subjects in CIS questionnaire.

With RiaGev supplementation, an alleviation in physiological signs of stress was observed via waking salivary cortisol relative to the Comparator. Supporting this finding were significant improvements, up to over 24%, in subjective fatigue, concentration, motivation and in total CIS scores with RiaGev.

RiaGev was found to be safe and well-tolerated in healthy adults between the ages of 35 to 65 years. Only two minor adverse events (weakness and appetite) were observed. Notably, there were no skin flushing related adverse events reported, a common side effect of NAD precursor supplements. Also no relevant changes were observed in clinical chemistry and hematology.

It is worth noting that no MeNAM and NAAD(P) were detected in the blood samples with RiaGev supplementation in the study. MeNAM and NAAD(P) are abundantly common by-products in NR and NAM supplementations. [12, 34] Combining D-ribose with NAM in RiaGev apparently reduced MeNAM and NAAD(P) formation. This is consistent with enhanced NAD+ metabolome production and potentially reduced side effects as demonstrated in the study. Thus, combining D-ribose with NAM provides a way to employ NAM in higher dose safely and effectively.

The human clinical investigation focused on healthy active population in midlife stage. Previous studies were focused either on the obese or on a senior population, where oxidative stress is not considered as an ongoing factor. [19, 34, 35] It is firmly established that oxidative stress is the most prevalent factor leading to disease and premature aging. [29, 30, 36] Some animal studies indicate that the other NAD+ enhancing ingredients, including NR, do not protect subjects from exercise-induced oxidative stress. On the contrary, NAD+ enhancing ingredients, including NR, contributes to greater oxidative stress by depleting NADPH and glutathione. [21, 22]. This is potentially serious since greater oxidative stress is an integral part of daily life for every active person, especially in their midlife period. The study demonstrates that RiaGev enhances energy and glutathione levels, protecting subjects from oxidative injury. This is clearly demonstrated in the preservation of redox homeostasis during exhaustive aerobic exercise as well as steady and lower salivary cortisol in the RiaGev Group as compared to a higher and fluctuating salivary cortisol in the Comparator Group.

Oxidative stress is not only induced by daily activities, it may also be induced by some food and beverage that we consume every day, primarily from the ingestion of high glycemic index diets that lead to glucose intolerance and insulin resistance. RiaGev has not been found to acutely reduce blood glucose peak after a meal as D-ribose alone has been shown to do. [37] On the contrary, RiaGev enhances the clearance of glucose from blood stream so that the glucose peak diminishes more quickly. More importantly, this overall blood glucose reduction is achieved without overall more insulin secretion. This suggests that RiaGev improves insulin sensitivity and glucose intolerance. The result is particularly relevant because the subjects in the RiaGev Group have a high hemoglobin (HbA1c) to start with. HbA1c reflects average daily glucose level in the blood. Average HbA1c of 5.5% in the RiaGev Group is typical for persons around 50 years of age in the US, which is also close to the upper limit (=5.7%) for healthy populations. Significant reduction of overall blood glucose by RiaGev supplementation in this population is particularly important for scientific as well as for practical implications.

Although successful, the clinical trial had its limitations. One obvious limitation was its relatively short duration. Since this was the first clinical trial for RiaGev, it was designed based on Applicant's preclinical animal studies, which are usually short in duration. The short duration made the last clinical visit, i.e. Day 8, too full of activities that it pushed the sampling into the afternoon, far behind the sampling time on Day 1, Day 3, and 5. This sampling time difference is believed to be the main reason that measurements on Day 8 did not follow the trend of Day 3 and Day 5, resulting in lower than expected measurement in NAD+ metabolome as well as from the CIS questionnaire. Previous studies indicate the NAD+ metabolome is highly regulated by circadian rhythm and the afternoon typically has lower levels of NAD+ metabolome.

The CIS scores of both RiaGev and Comparator Group improved after Day 1 during the testing period. This is likely due to the fact that subject had been required to keep a food and sleep dairy during the study period, which result in more regularity in diet and sleep, which in turn improved in blood glucose as well as CIS scores in all participants.

The blood glucose and insulin levels of the RiaGev Group are consistently higher than that of Comparator Group, especially at Day 1 baseline. This is a reflection that RiaGev-Comparator and Comparator-RiaGev sequence groups are not very well matched in this aspect. Glycosylated Hemoglobin (HbA1c) level in the first RiaGev-Comparator Group was 5.50% vs 5.25% in the Comparator-RiaGev Group (p=0.108), which translates into more than 7 mg/dL of average blood glucose in the RiaGev Group than the Comparator Group to start with. After 7-day supplementation, overall blood glucose in RiaGev Group reduced to a significant extent (61%, p=0.013), that the overall blood glucose at Day 8 is essentially the same between the two groups. This is a strong indication that RiaGev significantly improves blood glucose level.

Due to the favorable safety profile of RiaGev and its strong improvement in NAD+ metabolomes and blood glucose, future work should expand the population to subjects at risk of reduced NAD+ levels, such as the elderly, those with impaired glucose tolerance, and those suffering from the intricate aspects of metabolic syndrome, including prediabetes and diabetes. More generally, people suffering from oxidative stress would benefit from RiaGev. Compared to NR and NMN [38], NAM is inexpensive and has a long history of safety [12]. The combination of D-ribose and NAM in RiaGev improves NAM metabolism, which may improve human performance [39], and which can provide a new approach to enhance the NAD+ metabolome. On the other hand, the excellent safety profile of RiaGev suggests that combination of D-ribose with nicotinamide may provide a new approach to expand the usage and dosage of this vitamin B3 for human benefits.

While the RiaGev used in the clinical trial contained an effective amount of nicotinamide and D-ribose with an optimized ratio of nicotinamide to D-ribose of approximate 1 to 5 which achieved the results identified above, it is anticipated that an effective amount of nicotinamide and D-ribose over a wide range of ratios of nicotinamide to D-ribose and a wide range of dosages, will increase NAD levels in human subjects, along with an increase in glutathione levels without causing redox unbalance. For example, as disclosed in International Patent Application No. PCT/US2019/031889 (Publication No. WO2019/217935), incorporated herein by reference, the effective amount of nicotinamide and D-ribose may have ratios between 0.5:10 and 10:0.5 nicotinamide to D-ribose or between 1:5 and 5:1 of nicotinamide to D-ribose and the effective amount of nicotinamide and D-ribose may be between 20 mg to 5400 mg per day or between 100 mg to 4000 mg per day.

### CONCLUSION

The randomized, triple blind, comparator-controlled, crossover pilot study assessed the efficacy and safety of a combination of nicotinamide and D-ribose (RiaGev®) in healthy adults. Supplementation with RiaGev effectively increased concentrations of NAD+ metabolome, especially NADP+ level, in circulating blood. It also enhanced the high energy phosphate and glutathione levels in the blood. The RiaGev Group had significantly improved post-prandial glucose tolerance. The circulating antioxidants, including GSH and NADPH, were also enhanced with RiaGev. This aspect is more pronounced with exhaustive aerobic exercise, where RiaGev preserves redox homeostasis overwise disturbed by the oxidative stress. The waking cortisol, a stress hormonal, was also consistently lower in the RiaGev Group than in the Comparator Group. The CIS questionnaire assessment indicates that RiaGev reduced physical fatigue, improved concentration, motivation, as well as overall well-being of the subjects. In summary, RiaGev was found to be safe and well-tolerated in healthy adults and its favorable safety profile suggests that the combination of D-ribose with nicotinamide may help to expand the usage of this vitamin B3 for broad applications and human benefits.

### REFERENCES

1. Houtkooper R H., Canto C., Wanders R. J., Auwerx J. 2010. The secret life of nad(+): an old metabolite controlling new metabolic signaling pathways. Endocr Rev. 31:194-223.
2. Satoh A., Stein L., Imai S. 2011. The role of mammalian sirtuins in the regulation of metabolism, aging, and longevity. Handb Exp Pharmacol. 206:125-162
3. Imai S., Yoshino J. 2013. The importance of NAMPT/NAD/SIRT1 in the systemic regulation of metabolism and aging. Diabetes Obes Metab. 15(3):26-33.
4. Nakahata Y., Sahar S., Astarita G., Kaluzova M., Sassone-Corsi P., 2009. Circadian control of the nad+ salvage pathway by clock-sirt1. Science. 324:654-657.
5. Evans C., Bogan K. L., Song P., Burant C. F., Kennedy R. T., et al., 2010. Nad+ metabolite levels as a function of vitamins and calorie restriction: evidence for different mechanisms of longevity. BMC Chem Biol. 10:2.
6. Belenky P., Bogan K. L., Brenner C. 2007. NAD+ metabolism in health and disease. Trends Biochem Sci. 32:12-19.
7. Canto C., Houtkooper R. H., Pirinen E., Youn D. Y., Oosterveer M. H., et al. 2012. The nad(+) precursor nicotinamide riboside enhances oxidative metabolism and protects against high-fat diet-induced obesity. Cell Metab. 15:838-847.
8. Mouchiroud, L.; Houtkooper, R. H.; Auwerx, J. 2013. NAD(+) metabolism: a therapeutic target for age-related metabolic disease. Crit Rev Biochem Mol Biol. 48:397-408.
9. Nikiforov A., Dolle C., Niere M., Ziegler M. 2011. Pathways and subcellular compartmentation of nad biosynthesis in human cells: from entry of extracellular precursors to mitochondrial nad generation. J Biol Chem. 286:21767-21778.
10. Rajman L., Chwalek K., Sinclair D. A. 2018. Therapeutic potential of NAD-boosting molecules: the in vivo evidence. Cell Metab. 27(3):529-547.
11. Stavrum A. K., Heiland I., Schuster S., Puntervoll P., Ziegler M. 2013. Model of tryptophan metabolism, readily scalable using tissue-specific gene expression data. J Biol Chem. 288(48):34555-34566.
12. Mackay, D.; Hathcock, J.; Guarneri, E. 2012. Niacin: chemical forms, bioavailability, and health effects, Nutr Rev. 70:357-366.
13. Imai S. 2009. Nicotinamide phosphoribosyltransferase (nampt): A link between nad biology, metabolism, and diseases. Curr Pharm Des. 15:20-28.
14. Imai S., Yoshino J. 2013. The importance of NAMPT/NAD/SIRT1 in the systemic regulation of metabolism and aging. Diabetes Obes Metab. 15(3):26-33.
15. Aman, Y.; Qiu, Y.; Tao, J.; Fang, E. F. 2018. Therapeutic potential of boosting NAD+ in aging and age-related diseases. Transl Med Aging. 2:30-37.
16. De Guia R. M., Agerholm M., Nielsen T. S., Consitt L. A., Sogaard D., Helge J. W., Larsen S., Brandauer J., Houmard J. A., Treebak J. T. 2019. Aerobic and resistance exercise training reverses age-dependent decline in nad(+) salvage capacity in human skeletal muscle. Physiol Rep. 7(12):e14139.
17. Trammell S. A., Schmidt M. S., Weidemann B. J., Redpath P., Jaksch F., Dellinger R. W., Li Z., Abel E. D., Migaud M. E., Brenner C. 2016. Nicotinamide riboside is uniquely and orally bioavailable in mice and humans. Nat Commun. 7:12948.
18. Ratajczak J., et al., 2016. Nrk1 controls nicotinamide mononucleotide and nicotinamide riboside metabolism in mammalian cells. Nat Commun. 7:13103
19. Dollerup, O. L.; Christensen, B.; Svart, M.; Schmidt, M. S.; Sulek, K.; Ringgaard, S.; Stødkilde-Jørgensen, H.; Moller, N.; Brenner, C.; Treebak, J. T.; Jessen, N. 2018. A randomized placebo-controlled clinical trial of nicotinamide riboside in obese men: safety, insulin-sensitivity, and lipid-mobilizing effects. Am J Clin Nutr. 108(2):343353
19. Liu L., Su X., Quinn W. J. 3rd, Hui S., Krukenberg K., Frederick D. W., Redpath P., Zhan L., Chellappa K., White E., Migaud M., Mitchison T. J., Baur J. A., Rabinowitz J. D. 2018. Quantitative Analysis Of Nad Synthesis-Breakdown Fluxes. Cell Metab. 27(5):1067-1080.
20. Campbell M. T. D., Jones D. S., Andrews G. P., Li S. 2019. Understanding the physicochemical properties and degradation kinetics of nicotinamide riboside, a promising vitamin B(3) nutritional supplement. Food Nutr Res. 63.
21. Kourtzidis I. A., Stoupas A. T., Gioris I. S., Veskoukis A. S., Margaritelis N. V., Tsantarliotou M., Taitzoglou I., Vrabas I. S., Paschalis V., Kyparos A., Nikolaidis M. G. 2016. The nad+ precursor nicotinamide riboside decreases exercise performance in rats. J Int Soc Sports Nutr. 13:32.
22. Kourtzidis I. A., Dolopikou C. F., Tsiftsis A. N., Margaritelis N. V., Theodorou A. A., Zervos I. A., Tsantarliotou M. P., Veskoukis A. S., Vrabas I. S., Paschalis V., Kyparos A., Nikolaidis M. G. 2018. Nicotinamide riboside supplementation dysregulates redox and energy metabolism in rats: implications for exercise performance. Exp Physiol. 103(10):1357-1366.
23. Goldsmith G. A., Sarett, H. P., Register U. D., Gibbens J. 1952. Studies of niacin requirement in man. I. Experimental pellagra in subjects on corn diets low in niacin and tryptophan. J Clin Invest. 31(6):533542.
24. Chen, A. C.; Martin, A. J.; Choy, B.; Fernandez-Pefias, P.; Dalziell, R. A.; McKenzie, C. A.; Scolyer, R. A.; Dhillon, H. M.; Vardy, J. L.; Kricker, A.; St George, G.; Chinniah, N.; Halliday, G. M.; Damian, D. L. 2015. A phase 3 Randomized Trial of Nicotinamide for Skin-Cancer Chemoprevention. N Engl J Med. 373(17):1618-1626.
25. Bogan, K. L.; Brenner, C. 2008. Nicotinic Acid Nicotinamide and Nicotinamide Riboside: A Molecular Evaluation of NAD(+) Precursor Vitamins in Human Nutrition. Annu Rev Nutr. 28:115-130.
26. Knip M., Douek I. F., Moore W. P., Gillmor H. A., McLean A. E., Bingley P. J., et al. 2000. Safety of high-dose nicotinamide: A review. Diabetologia 43 (11):1337-1345.
27. Centers for Disease Control and Prevention, National Center for Health Statistics (NCHS); National Health and Nutrition Examination Survey Data; Hyattsville, M.D.: U.S. Department of Health and Human Services, Centers for Disease Control and Prevention, Https://wwwn.cdc.gov/nchs/nhanes/default.aspx (2011-2016).
28. Winter S. L., Boyer J. L. 1973. Hepatic toxicity from large doses of vitamin B3 (nicotinamide). N Engl J Med. 289(22):1180-1182.
29. Soysal P., Isik A. T., Carvalho A. F., Fernandes B. S., Solmi M., Schofield P., Veronese N., Stubbs B. 2017. Oxidative stress and frailty: A systematic review and synthesis of the best evidence. Maturitas. 99:66-72.
30. Bonomini F., Rodella L. F., Rezzani R. 2015. Metabolic Syndrome, Aging and Involvement of Oxidative Stress. Aging Dis. 6(2):109-120.
31. Nagana Gowda, G. A.; Abell, L.; Lee, C. F.; Tian. R.; Raftery, D. 2016. Simultaneous Analysis of Major Coenzymes of Cellular Redox Reactions and Energy Using ex Vivo (1)H NMR Spectroscopy. Anal Chem. 4788(9):4817-4824.
32. Vercoulen, J. H.; Swanink, C. M.; Fennis, J. F.; Galama, J. M.; van der Meer, J. W.; Bleijenberg, G. 1994. Dimensional assessment of chronic fatigue syndrome. Journal of Psychosomatic Research. 38(5):383-392.
33. This clinical trial is registered at ClinicalTrial.gov. NCT number 04483011.
34. Airhart S. E., Shireman L. M., Risler L. J., Anderson G. D., Nagana Gowda G. A., Raftery D., et al. 2012. An open-label, non-randomized study of the pharmacokinetics of the nutritional supplement nicotinamide riboside (NR) and its effects on blood NAD+ levels in healthy volunteers. PloS One. 12:e0186459.
35. Conze D., Brenner C., Kruger C. L. 2019. Safety and Metabolism of Long-term Administration of NIAGEN (Nicotinamide Riboside Chloride) in a Randomized, Double-Blind, Placebo-controlled Clinical Trial of Healthy Overweight Adults. Sci Rep. 9(1):9772.
36. Massudi H., et al. 2012. Age-associated changes in oxidative stress and nad(+) metabolism in human tissue. Plos One. 7:e42357.
37. Seifert, J.; Frelich, A.; Shecterle, L.; St Cyr, J. 2008. Assessment of Hematological and Biochemical parameters with extended D-Ribose ingestion. J Int Soc Sports Nut. 15(5):13
38. Yoshino J., Mills K. F., Yoon M. J., Imai S. 2011. Nicotinamide mononucleotide, a key nad(+) intermediate, treats the pathophysiology of diet-and age-induced diabetes in mice. Cell Metab. 14:528-536.
39. Bitterman K J, Anderson R. M., Cohen H Y, Latorre-Esteves M., Sinclair D A. 2002. Inhibition of silencing and accelerated aging by nicotinamide, a putative negative regulator of yeast sir2 and human sirt1. Biol Chem. 277:45099-45107.

1. A method of increasing NAD metabolome in human subjects by administering to the subjects an effective amount of D-ribose and nicotinamide.

2. The method of claim 1, wherein NAD metabolome and glutathione levels are increased in the subjects.

3. The method of claim 2, wherein NAD metabolome and glutathione levels are increased without causing redox unbalance in the subjects.

4. The method of any one of claims 1 to 3, wherein the effective amount is a ratio between 0.5:10 and 10:0.5 of nicotinamide to D-ribose.

5. The method of any one of claims 1 to 3, wherein the effective amount is a ratio between 1:5 and 5:1 of nicotinamide to D-ribose.

6. The method of any one of claims 1 to 5, wherein the effective amount is between 20 mg to 5400 mg per day.

7. The method of any one of claims 1 to 5, wherein the effective amount is between 100 mg to 4000 mg per day.

8. The method of any one of claims 1 to 7, wherein the effective amount is administered to the subjects in the morning and in the evening.

9. The method of claim 8, wherein the effective amount is administered to the subjects immediately before the subjects eat breakfast in the morning and immediately before the subjects eat dinner in the evening.

10. A composition administered to human subjects to increase NAD metabolome in human subjects, the composition comprising:
an effective amount of D-ribose and nicotinamide.

11. The composition of claim 10, wherein the composition increases NAD metabolome and glutathione levels in the subjects.

12. The composition of claim 11, wherein the composition increases NAD metabolome and glutathione levels in the subjects without causing redox unbalance in the subjects.

13. The composition of any one of claims 10 to 12, wherein the effective amount is a ratio between 0.5:10 and 10:0.5 of nicotinamide to D-ribose.

14. The composition of any one of claims 10 to 12, wherein the effective amount is a ratio between 1:5 and 5:1 of nicotinamide to D-ribose.

15. The composition of any one of claims 10 to 14, wherein the effective amount is between 20 mg to 5400 mg per day.

16. The composition of any one of claims 10 to 14, wherein the effective amount is between 100 mg to 4000 mg per day.

17. The composition of any one of claims 10 to 16, wherein the composition is administered to the subjects in the morning and in the evening.

18. The composition of claim 17, wherein the composition is administered to the subjects immediately before the subjects eat breakfast in the morning and immediately before the subjects eat dinner in the evening.
